(19) United States
(12) Patent Application Publication
(10) Pub. No.: US 2003/0028097 A1
(43) Pub. Date: Feb. 6, 2003

(54) Immobilizer probe system and method

(76) Inventors: Anthony V. D'Amico, Weston, MA (US); Robert Allan Cormack, Milton, MA (US); Donna Cressman-Feo, Poland, OH (US); Bradley J. Adams, Leechburg, PA (US)

Correspondence Address: Gregory L. Bradley, Medrad, Inc., One Medrad Drive, Indianola, PA, 15051, US

(21) Appl. No.: 10/211,480

(22) Filed: Aug. 2, 2002

Related U.S. Application Data
(60) Provisional application No. 60/309,859, filed on Aug. 3, 2001.

(51) Int. Cl. A61B 5/05

(52) U.S. Cl. 600/427

(57) ABSTRACT

A system including an insertable probe and method of use thereof for immobilizing a region of interest during staging and radiation therapy thereof. The probe in its preferred embodiment is for immobilizing the male prostate region during target localization using portal imaging and radiation therapy to treat prostate cancer and comprises an elongated shaft supporting an inflatable balloon.

### CROSS REFERENCE TO RELATED APPLICATION

This application claims the benefit of U.S. Provisional Application Serial No. 60/309,859, filed on Aug. 3, 2001, the contents of which are incorporated herein by reference.

### FIELD OF THE INVENTION

The present invention relates generally to a system and method for performing diagnostic and therapeutic treatments of disease. Specifically, the present invention relates to an insertable probe system and method of use thereof designed to immobilize the prostate region during target localization and radiation therapy to treat prostate cancer.

### BACKGROUND OF THE INVENTION

Treatment of prostate cancer using radiation therapy is difficult due to the prostate's position near radiation sensitive tissues, and is further complicated by prostate motion. Adenocarcinoma of the prostate commonly occurs in the posterior portion or the apex of the prostate gland. To plan external beam radiation treatment, urethrograms, CT and magnetic resonance imaging (MRI) have all been used to localize the prostatic apex.

U.S. Pat. No. 5,476,095 describes an insertable pickup probe for use in providing diagnostic MRI images. The pickup probe in its preferred embodiment is for use in imaging the male prostate and comprises an elongated shaft supporting an inflatable patient interface balloon at its distal end. The interface balloon comprises an inner balloon and an outer balloon, between which a receiving coil is positioned. A lumen for air supply is provided in the shaft for expanding the inner balloon against the outer balloon to place the receiving coil in close proximity to the area of interest in order to provide MRI images.

Typically, planning of radiation therapy for the treatment of prostate cancer involves the patient undergoing a CT based simulation scan of the pelvis to determine the location of the prostate gland. In the simulation phase, the patient is placed on CT equipment that is preferably similar to the radiation treatment equipment (except that it does not generate the high energy radiation beam). The simulation equipment is positioned to simulate the delivery of the sequence of treatment beams prescribed by the treating oncologist. During this procedure a low dosage x-ray image, called the simulation image, is taken. This simulation image helps the oncologist locate the position of the tumor and thereby establish the positions of the radiation equipment components for delivering the successive treatment beams.

During the actual treatment phase, the patient is placed in the same position on the treatment equipment as in the simulation scans . Radiation-emitting devices are generally known and used for radiation therapy in the treatment of patients. Typically, a radiation therapy device includes a gantry, which can be swiveled around a horizontal axis of rotation in the course of a therapeutic treatment. A linear accelerator is located in the gantry for generating a high-energy radiation beam for therapy. During treatment, the radiation beam is provided on one zone of a patient lying in the isocenter of gantry rotation. A further feature of radiation therapy involves portal images, which are commonly used in radiation therapy to verify and record the patient tumor location. Portal images include manual (film) and electronic images (EPI) taken before or after the treatment. Electronic portal images (EPI), when taken before the treatment, give the therapist the opportunity of correcting for minor patient positioning errors before treatment.

During external beam radiation therapy, radiation is directed primarily to the prostatic apex which is near the rectal wall. A misdirected radiation beam may perforate the rectal wall causing radiation proctitus (rectal bleeding) . This toxicity is related to the total radiation dose prescribed and the volume of the anterior rectal wall receiving a high (>70 Gy) radiation dose. A major factor limiting radiation oncologists' attempts to reduce the volume of the anterior rectal wall receiving a high radiation dose is the intrinsic motion of the prostate gland up to 5 mm in the anterior to posterior direction caused by rectal peristalsis. Accordingly, oncologists generally will add a margin to the radiation field in order to ensure that the entire prostate gland receives the prescription dose. This margin is typically on the order of 15 mm. As a consequence, radiation therapists commonly lower the desired radiation so as not to overexpose radiation sensitive proximal structures. However, this may lead to inadequate radiation treatment and local cancer recurrence.

One method of prostate immobilization during radiotherapy is described in Teh BS, Mai W, Uhl BM, et al. Intensity-modulated radiation therapy (IMRT) for prostate cancer with the use of rectal balloon for prostate immobilization: acute toxicity and dose-volume analysis. Int J Radiat Oncol Biol Phys 2001; 49:705-712; and Teh BS, Woo Sy, Butler EB. Intensity modulated radiation therapy; A new promising technology in radiation oncology. The Oncologist 1999; 4:433-442. A rectal catheter with an inflatable spherical balloon is utilized during the initial planning stages and the radiation treatments. In that study, however, daily verification of the balloon position was not performed using portal imagery to localize the prostate, and treatment was localized based on external skin markings. Further, the spherical shaped balloon may potentially deform and/or rock the prostate due to its shape. It also may be difficult to consistently control the depth of insertion so that the balloon is positioned against the prostate the same way each insertion.

Therefore, there is a need for a method and system for immobilizing the prostate gland in the same position for each radiation therapy session and thereby limiting the volume of the anterior rectal wall that would receive radiation through smaller posterior margins and allowing for the delivery of higher radiation doses.

There is a further need to position the anterior rectal wall away from the prostate gland during radiation therapy, thereby reducing radiation exposure.

There is a further need to visualize the prostate region using portal imaging before treatment to confirm the position of the prostate and to adjust for any changes in size of the prostate.

There is a further need for a prostate immobilizer balloon whose shape overcomes the deficiencies of prior balloons.

### SUMMARY OF THE INVENTION

The present invention in its most preferred embodiment relates to a radiation therapy using an insertable, intracavity probe, and more specifically an intrarectal probe and method of using the probe for immobilizing the prostate during staging purposes and radiation therapy treatment. Although the probes are described hereinafter as principally to image and immobilize the prostate, it should be understood that the concepts outlined herein are equally appropriate for other applications necessitating an imaged and immobilized area and for other regions of interest such as the vagina, or other parts of the body reachable by an insertable probe.

The immobilizer probe of the present invention comprises a shaft, which supports an inflatable patient interface balloon at its distal end. A lumen for air supply is provided in the shaft for expanding the balloon in close proximity to the region of interest once the immobilizer probe is inserted into the body of the patient. In the preferred embodiment of the present invention, the probe is a prostate probe and is designed for insertion into the body intrarectally. An anti-migration disc is optionally provided which fits onto the shaft of the probe to prevent migration of the probe superiorly during the normal peristaltic activity of the colon.

The immobilizer probe of the present invention allows for accurate longitudinal and radial positioning of the balloon within the body by making the shaft rigid when twisted radially. The balloon, shaft and handle are bonded together so that they move radially as a single unit when torque is applied. The distal tip of the probe is more flexible than the shaft to avoid perforating tissue during use. A syringe is preferably provided which connects to the shaft and functions as an air pump to deliver a volume-limited amount of air through the air lumen of the shaft to the balloon. Alternatively, other inflater devices such as an inflater cuff could be used. Furthermore, a stop cock is provided to maintain the air within the balloon. The probe along with the balloon, syringe, shaft and anti-migration disc are preferably disposable.

Preferably, the interface balloon of the present invention is sized and shaped substantially identically to the outer balloon of the insertable imaging probe of U.S. Pat. No. 5,476,095. However, the probe of the present invention is constructed without a receiving coil and an inner balloon. The saddle shape of the balloon of the present invention conformingly fits the rectal prostatic bulge inferior to the ampulla of the rectum whereby the prostate is neither deformed or rocked when the immobilizer probe is inserted. The saddle shape also allows each probe to be consistently inserted to the same depth relative to the prostate.

Initially, a diagnostic MRI is performed using the insertable imaging probe and procedure discussed in U.S. Pat. No. 5,476,095. Later, the immobilizer probe of the present invention is be inserted and inflated for use in a CT based simulation scan. A radiation treatment is planned based on the staging data. During the radiation therapy, the immobilizer probe of the present invention is inserted and inflated to the same amount as was done during staging. Portal images are taken before each treatment or at least spaced throughout the treatment regimen to verify the size and position of the prostate.

The immobilizer probe of the present invention has been found to decrease maximal prostate gland motion in the anterior-posterior direction from approximately 4 mm to 1 mm during the interval necessary to deliver two lateral radiation treatment fields. In addition, localization of the mucosal surface of the anterior rectal wall has been found to be within the maximal anterior-posterior displacement of the prostate gland (<1.0 mm) when the intra-rectal balloon is inflated. By using the portal image of the therapy device, the anterior surface of the intra-rectal balloon can define the anterior rectal wall to within 1 mm. This ensures that a posterior field margin set 1 mm posterior to the anterior surface of the balloon would deliver a full radiation dose to the entire prostate gland because 3-5 mm of tissue exists in the prostatic rectal interface to permit dosimetric build up.

Further, when androgen suppression therapy (AST) is given concurrently with external beam RT, it is known that during the course of RT the prostate gland will decrease in volume under the effect of AST, bringing more rectal mucosa into the high dose RT volume and thereby increasing the risk of rectal toxicity. Therefore, the present invention allows for adjustment of the posterior border of the lateral treatment fields due to the decreasing prostate volume during the course of RT. This helps to reduce rectal volume being treated to above 70 Gy while still ensuring dosimetric coverage of the entire prostate gland.

The present invention, along with the attributes and attendant advantages thereof, will best be appreciated and understood in view of the following detailed description taken in conjunction with the accompanying drawings.

### BRIEF DESCRIPTION OF THE DRAWINGS

In the accompanying drawings, a preferred embodiment of the present invention is illustrated, by way of example only, wherein:

FIG. 1 is a perspective view illustrating an imaging probe of the type disclosed in U.S. Pat. No. 5,476,095.

FIG. 2 is a cross-sectional view taken through line 2-2 of the distal inflatable balloon portion of the imaging probe illustrated in FIG. 1.

FIG. 3 is an end view as seen from line 3-3 of the imaging probe illustrated in FIG. 1.

FIG. 4 is a sectional view taken through line 4-4 of FIG. 2.

FIG. 5 is a top sectional view as seen from line 5-5 of FIG. 2.

FIG. 6 is a cross-sectional view illustrating the shaft of the imaging probe of FIG. 1.

FIG. 7 is a perspective view illustrating the immobilizer probe of the present invention.

FIG. 8 is a cross-sectional view taken through line 8-8 of the distal inflatable balloon portion of the immobilizer probe illustrated in FIG. 7.

### DETAILED DESCRIPTION OF THE INVENTION

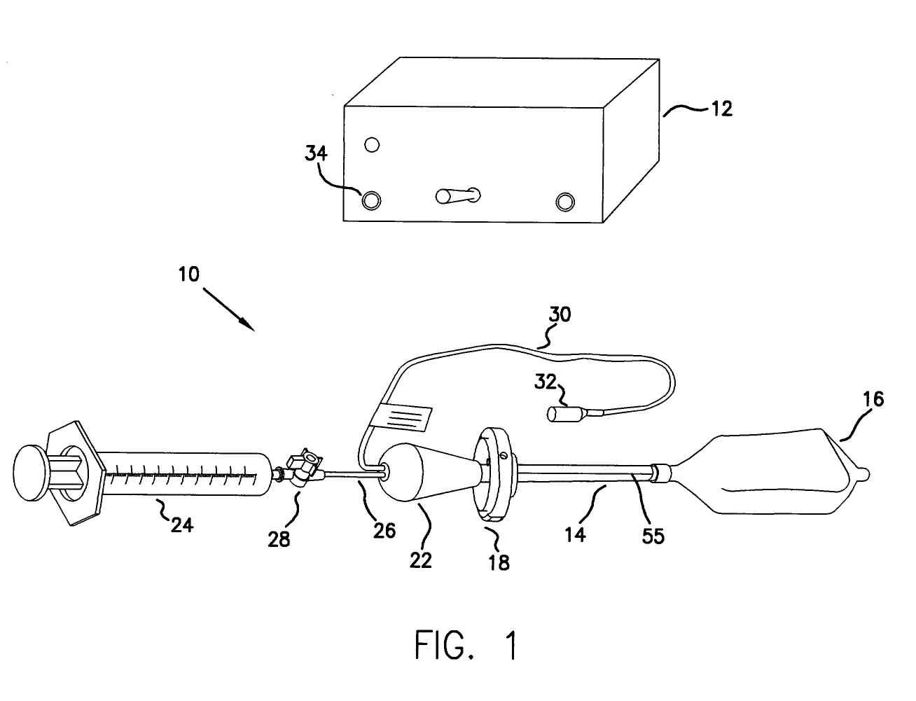

Referring first to FIG. 1, an insertable prostate imaging probe of the type disclosed in U.S. Pat. No. 5,476,095 is shown in an assembled form at 10, which connects to an interface network 12. The insertable prostate imaging probe 10 is an MRI receiving device capable of imaging or gathering spectra from the human prostate and surrounding tissue, but may also be used as the transmit coil for RF excitation. The probe 10 is used with the interface network 12 which provides the tuning, impedance matching, and decoupling functions.

The probe 10 includes a shaft 14 which supports a patient interface balloon at its distal end, an anti-migration disc 18, and a handle 22 located at the proximal end of the shaft 14. A syringe 24 is provided for supplying air to the patient interface balloon 16 and connects to the proximal end of the shaft by a tube 26. A stop cock 28 is provided in the tube 26 for controlling the passage of air through the tube 26 to the patient interface balloon 16.

A receiving coil is contained within the patient interface balloon 16 and electrically connected to the interface 12 by an insulated interconnecting cable 30 which has a plug 32 at its proximal end for connection to terminal 34 located on the front of the interface network 12.

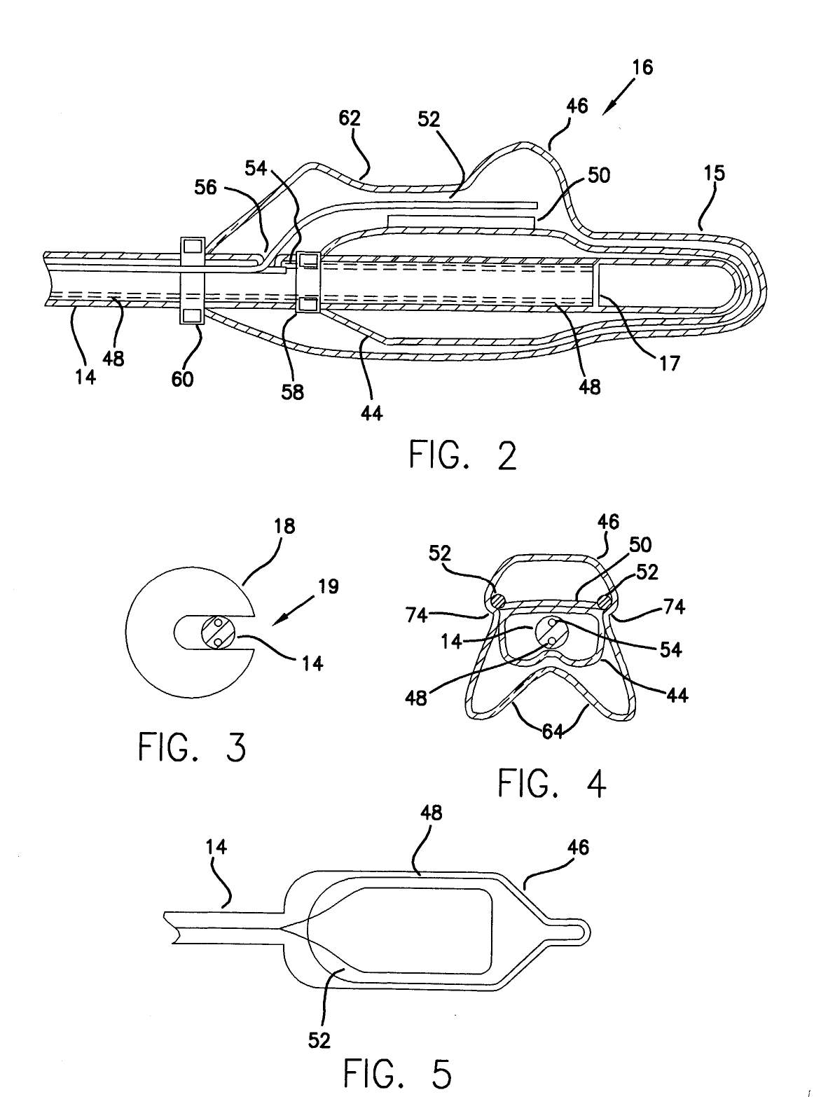

Referring now to FIGS. 2, 4, 5 and 6, the patient interface balloon 16 of the insertable imaging probe 10 is illustrated in more detail. The patient interface balloon 16 comprises an inner balloon 44 and an outer balloon 46. The inner balloon 44 is constructed of a flexible medical grade latex or other elastomeric material, which is preferably non-paramagnetic and has low dielectric losses, and is capable of being inflated with air supplied through a lumen 48 within the shaft 14, and expelled into the inner balloon 44 via hole 49 in lumen 48. The inner balloon 44 is substantially cylindrical in shape except for an anterior substantially planar section which is covered with a non-stretchable substantially planar member 50, formed, of for example, an adhesive backed cloth material.

A receiving coil 52 is provided between the inner balloon 44 and the outer balloon 46 and is typically formed of a flexible conductive material. The receiving coil 52 is arranged between the non-stretchable member 50 and the outer balloon 46, is fed to the patient interface balloon 16 through a second lumen 54 in the shaft 14, and is fed out of the shaft 14 through a hole 56 in the shaft 14 inside the outer balloon 46.

The outer balloon 46 has a first wall positioned adjacent the region of interest, which can have an anterior saddle shape as indicated at reference number 62, for conformably fitting the rectal prostatic bulge inferior to the ampulla of the rectum. In addition, the outer balloon 46 has a second wall position generally comprised by posterior undulating folds 64 which allow the patient interface balloon 16 to unfold first when the inner balloon 44 is inflated. That is, the second position of the outer balloon moves preferentially with respect to the first position so that it can move into contact with a vessel wall generally opposite the region of interest during the initial stages of inflation of inner balloon 44. This unfolding forces the anterior surface 62 to hug the prostatic region of the rectum, thereby ensuring that that the image field of view of the insertable imaging probe 10 will focus on the desired region of interest.

The non-stretchable member 50 serves two functions in the patient interface balloon 16. First, the member 50 controls the focus of the inflation stretch of the inner balloon 44; secondly, member 50 acts as a guide for the receiving coil 52. Upon inflation, the inner balloon 44 first stretches posteriorly away from the receiving coil 52. This initiates the folds 64 of the outer balloon 46 to force posteriorly against the rectum wall until the anatomy offers an equal resistance. Then, the non-stretchable member 50 rises and forces the receiving coil 52 and the anterior surface 62 of the outer balloon 46 against the region of interest. When inflation is complete, the receiving coil 52 is in position to receive the best possible RF signal from the region of interest.

In addition, as shown in FIG. 4, lateral indentations 74 are provided on the outer balloon 46 intermediate the first and second wall positions. The indentations 74 act as coil positioners when the balloon is in its uninflated state. The receiving coil 52 is positioned on the shelf formed by the indentations 74 during assembly of the probe. This allows the receiving coil 52 to be repeatedly positioned relative to the shelf inside the outer balloon 46 for numerous clinical inflation and deflation cycles.

Alternatively, the patient interface balloon 16 may be constructed with a single ply inflatable balloon of elastomeric material. In this arrangement, the receiving coil 52 would be bonded to the inside surface of the balloon.

Further yet, the interface balloon 16 may be constructed with a single multi-ply balloon. This balloon would have the receiving coil 52 encapsulated between the plies of the elastomeric material. When inflated, the receiving coil 52 would be forced against the region of interest by the movement of the balloon. The coil encapsulation would take place during the balloon fabrication process by placing the receiving coil 52 on the surface of the balloon and then redipping the balloon to place another ply of material over the outer surface of the balloon, thus covering the receiving coil 52.

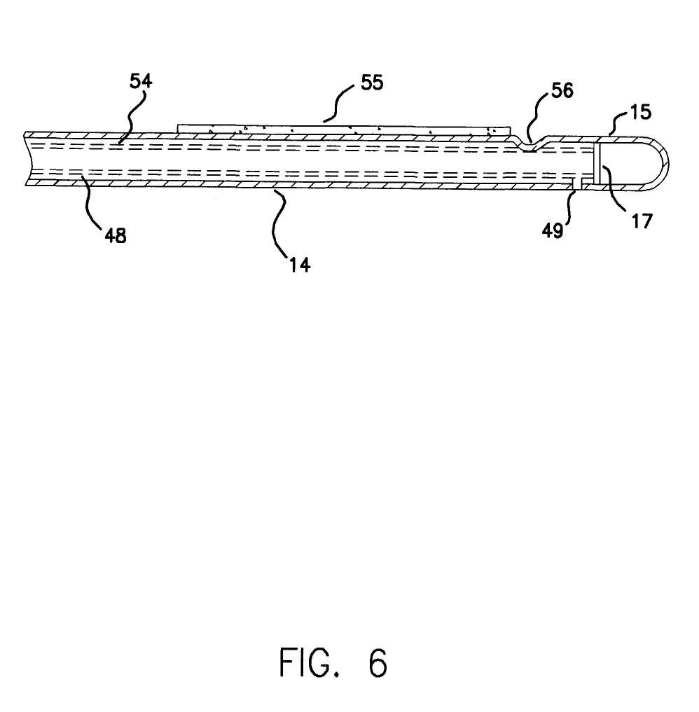

To assist a clinician in the insertion of the pickup probe 10, a colored stripe 55 is painted or otherwise marked on the shaft 14. The stripe 55, best shown in FIG. 1, and also shown in FIG. 6, may include a scale for indicating the distance which the shaft 14 has been inserted in the patient, and also the radial orientation of the balloon 16 for proper alignment with the prostate. In addition, the distal end 15 (hereinafter referred to as the flexible tip), of the shaft 14 which fits into the balloon 16 is typically more flexible than the remaining length of the shaft 14 to provide a more comfortable fit in the patient and to reduce the possibility of perforating tissue during use.

Referring to FIGS. 1, 2 and 6, the shaft 14 is rigid so that when it is twisted radially at the handle 22, the balloon, shaft, and handle move as a unit to ensure alignment. The flexible tip 15 is typically made of a more flexible material than the shaft 14, and is bonded to the shaft 14 as indicated at reference numeral 17. The outer balloon 62 is anchored to the shaft 14 by a proximal clamp 60 and by an interference fit with the flexible tip 15 of the shaft 14.

Similarly, the inner balloon 44 is anchored to the shaft 74 by a proximal clamp 58 and by an interference fit with the flexible tip 15. The outer balloon 46 completely encloses the flexible tip 15 of shaft 14 and inner balloon 44, as disclosed in FIG. 2.

FIG. 3 illustrates the anti-migration disc 18 in more detail as it fits onto the shaft 14. The disc 18 is semi-spherical and constructed from semi-rigid plastic. The purpose of the anti-migration disc 18 is to prevent the imaging probe 19 from migrating superiorly due to the normal peristaltic activity of the colon. The disc 18 has a slot 19 which snaps onto the shaft, as shown in FIG. 3, adjacent the anal sphincter after the device has been operatively placed within the patient.

In operation, the probe 10 is inserted intrarectally while the patient interface balloon 16 is in the uninflated relaxed state. The provided alignment guide 55 is used to radially and logitudinally position the probe 10 within or adjacent the region of interest.

The patient interface balloon 16 is then inflated via the syringe 24 to optimize the tissue to probe interface. The anti-migration disc 18 is then used to maintain proper positioning of the imaging probe 10 during the clinical scanning procedure.

Once the patient interface balloon 16 is inflated, the stop cock 28 is moved to a closed position, thus allowing the clinician to disconnect the syringe 24 without deflating the interface balloon 16. The probe 10 is then connected to the interface network 12 via plug 32 of the cable 30.

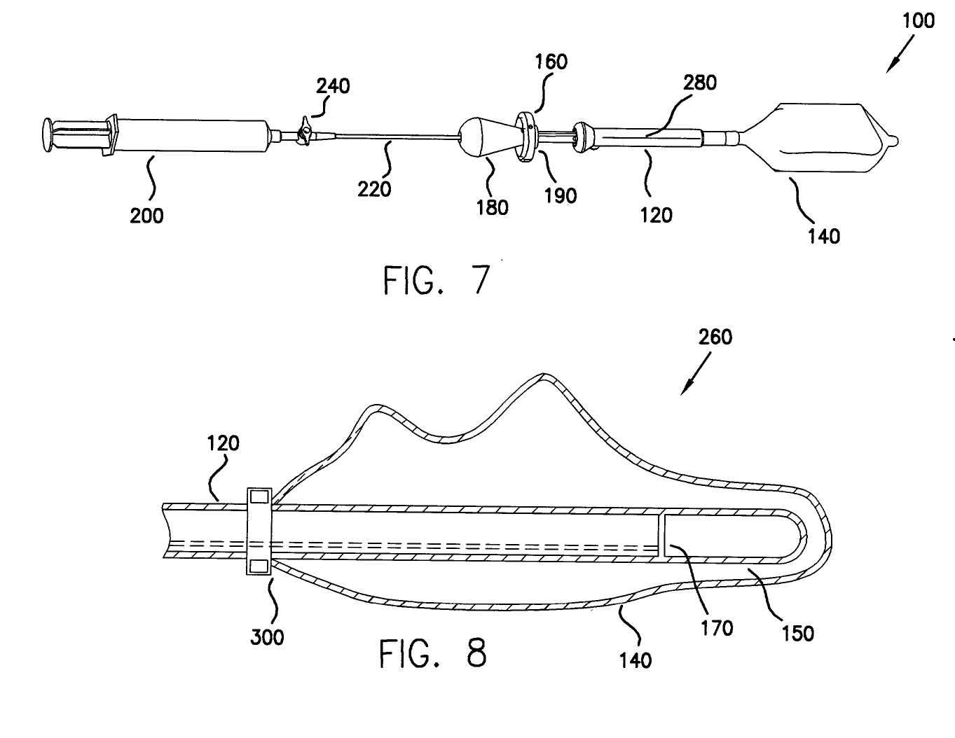

Referring now to FIG. 7, the immobilizer prostate probe 100 includes a shaft 120 which supports a patient interface balloon 140 at its distal end, an anti-migration disc 160 and a handle 180 located at the proximal end of the shaft. A syringe 200 is provided for supplying air to the patient interface balloon 140 and connects to the proximal end of the shaft 120 by a tube 220. A stop cock 240 is provided in the tube 220 for controlling the passage of air through the tube 20 to the patient interface balloon 140. The shaft 120 is provided with a lumen 130 within the shaft between tube 220 for providing passage of air to the balloon 140.

The interface balloon 140, best shown in FIG. 8 and substantially similar in shape to the outer balloon of the imaging probe discussed above, has a first wall positioned adjacent the region of interest, which can have an anterior saddle shape as indicated at reference 260, for conformingly fitting the rectal prostatic bulge inferior to the ampulla of the rectum. Upon inflation the balloon forces posteriorly against the rectum wall until the anatomy offers an equal resistance, thereby substantially immobilizing the prostatic region.

To assist a clinician in the insertion of the immobilizer probe, a colored stripe 280 is painted or otherwise marked on the shaft 120. The stripe 280, best shown in FIG. 7, may also include a scale for indicating the distance which the shaft has been inserted into the patient and the radial orientation of the balloon for proper alignment with the prostate. In addition, the distal end 150 of the shaft 120 which fits into the balloon 140 is typically more flexible than the remaining length to provide a more comfortable fit in the patient and to reduce the possibility of perforating tissue during use.

The shaft 120 is rigid so that when it is twisted radially at the handle 180, the balloon 140, shaft 120 and handle 180 move as a unit to ensure alignment. The flexible tip 150 is typically made of a more flexible material than the shaft 120 and is bonded to the shaft as indicated at reference numeral 170. The balloon 140 is anchored to the shaft 120 by a proximal clamp 300 and by an interference fit with the flexible tip 150. The balloon completely encloses the flexible tip 150 of shaft 120.

The anti-migration disc 160 is semi-circular and constructed from semi-rigid plastic. The purpose of the anti-migration disc 160 is to prevent the immobilizer probe from migrating superiorly. The disc 160 has a slot 190 which snaps onto the shaft 120 adjacent the anal sphincter after the device has been operatively placed within the patient. Upon completion of the intended purpose, the probe is deflated and then removed from the patient.

A preferred system and method utilizing the immobilizer probe will be described in more detail hereinafter. Initially, a diagnostic MRI is performed using the insertable imaging probe illustrated in FIGS. 1-6 and the procedure discussed in U.S. Pat. No. 5,476,095. Later, the immobilizer probe of the present invention (FIGS. 7 and 8) is inserted and inflated for use in a CT based simulation scan. The inflatable balloon portion of the immobilizer probe used during the simulation scan is filled with the same amount of air as that used in the imaging probe during the diagnostic MRI. CT images are obtained for approximately 5-10 minutes with the immobilizer probe inflated. The immobilizer probe is deflated and then removed from the patient.

A radiation treatment is planned based on the staging data including the size and location of the tumor. During the radiation therapy, the patient is placed in the exact same position on the equipment as the simulation and the immobilizer probe of the present invention is inserted to the same depth (e.g., 12 cm) and inflated (e.g., 60 cc) to the same degree as was done during the simulation. Portal images are taken before each treatment or at least spaced throughout the treatment regimen to verify the position of the prostate. The portal images are taken by delivering an image dose by the radiation emitting device. Because the balloon wall itself is not able to be “seen” in the portal images, the prostate is located by identifying the air cavity within the immobilizer probe balloon.[ Further, the anterior border of the balloon that is visualized in the portal image can be used to identify the mucosal surface of the anterior rectal wall to within 1 mm. This enables the posterior field border to be adjusted daily prior to treatment. This final adjustment contributes to minimizing the volume of rectal wall receiving a higher dose by allowing a smaller margin for set up error. This is of particular significance to patients receiving hormonal therapy concurrently to RT as prostate volume will decrease during the course of RT.

In the RT phase, high energy photons (>6 MV) and multiple fields (>4)are utilized for treatment. Radiation treatment is delivered by a therapeutic dose from the radiation emitting device with the immobilizer probe in place at the same depth and inflation volume as the staging and simulation. The prostate is treated initially in 15 treatments delivered at 180 cGy/fraction per dose for a total of 2700 cGy. This is followed by a treatment to the prostate and seminal vesicles consisting of 25 treatments delivered at 180 cGy/fraction per dose for a total of 4500 cGy. Margins around the Clinical Tumor Volume (CTV) are 5 mm for the first 15 treatments during which the prostate is the CTV and then 15 for the remaining 25 treatments during which the prostate and seminal vesicles are the CTV. Daily set up of the radiation therapy or cone down is performed using either portal film or imaging.

The immobilizer probe system and method of the present invention not only provides for the immobilization of the prostate during RT but also provides a continual and consistent means for localizing the prostate throughout the RT regimen. The present invention also provides the tracking of the size and location of the prostate which may change due to co-administered hormonal therapy. The present invention further provides for target verification through portal imaging prior to and throughout the RT regimen. It should be understood the concepts outlined herein regarding target verification are equally appropriate for other applications such as RT involving brain tumors during which the patient's skull is immobilized with respect to the table.

It is desirable to use a different immobilizer probe for each radiation treatment as potential exists for damaging the probe material by the radiation used in the RT.

The present invention also contemplates that the interface balloon be filled with a substance other than air such as contrast fluid to improve visibility of the balloon region.

If not otherwise stated herein, it may be assumed that all components and/or processes described heretofore may, if appropriate, be considered to be interchangeable with similar components and/or processes disclosed elsewhere in the specification, unless an indication is made to the contrary.

It should be appreciated that the apparatus and methods of the present invention may be configured and conducted as appropriate for the application. The embodiments described above are to be considered in all respects only as illustrative and not restrictive. The scope of the invention is defined by the following claims rather than by the foregoing description. All changes which come within the meaning and range of equivalency of the claims are to be embraced within their scope.

What is claimed is:

1. A method of planning radiation therapy treatment to an internal organ of a patient by a radiation emitting device, comprising:
substantially immobilizing the internal organ; and
acquiring a portal image of the patient by delivering an image dose from the radiation emitting device.

2. The method of claim 1, wherein the step of substantially immobilizing the internal organ comprises inserting a probe into a predetermined position in a cavity of the patient.

3. The method of claim 2, wherein the step of substantially immobilizing the internal organ further comprises inflating an interface device of the probe into contact with the cavity.

4. The method of claim 3, wherein the step of substantially immobilizing the internal organ further comprises causing an anterior saddle shaped surface of the interface device to conformingly fit against a shaped portion of the cavity proximate the internal organ.

5. The method of claim 4, wherein the internal-organ comprises a prostate gland.

6. The method of claim 1, wherein the steps of immobilizing the internal organ and providing an image dose are performed before the providing of a therapeutic dose by the radiation emitting device.

7. The method of claim 1, wherein the steps of immobilizing the internal organ and providing an image dose are performed before each delivery of a therapeutic dose by the radiation emitting device.

8. A method of planning and delivering radiation therapy treatment to an internal organ of a patient by a radiation emitting device, comprising:
substantially immobilizing the internal organ;
acquiring a portal image of the patient by delivering an image dose from the radiation emitting device; and
delivering a therapeutic dose from the radiation emitting device to the location of the internal organ based on the portal image.

9. The method of claim 8, wherein the step of substantially immobilizing the internal organ comprises inserting a probe into a predetermined position in the cavity of the patient.

10. The method of claim 9, wherein the step of substantially immobilizing the internal organ further comprises inflating an interface device of the probe into contact with the cavity.

11. The method of claim 10, wherein the step of substantially immobilizing the internal organ further comprises causing an anterior saddle shaped surface of the interface device to conformingly fit against a shaped portion of the cavity proximate the region of interest.

12. The method of claim 11, wherein the internal organ comprises a prostate gland.

13. An insertable intracavity probe for immobilizing an internal organ within a cavity of a patient during delivery of a dose from a radiation emitting device, the probe comprising:
a shaft member having a distal tip;
an interface device connected to the distal tip for conformingly fitting a bulge within the cavity corresponding to the internal organ to immobilize the internal organ during delivery of a dose from a radiation emitting device.

14. The probe of claim 13, wherein the interface device comprises an inflatable balloon.

15. The probe of claim 14, wherein the inflatable balloon defines a volume of air that is identifiable when subject to an image dose by the radiation emitting device to approximate the location of the internal organ.

16. The probe of claim 15, wherein the inflatable balloon comprises a surface having an anterior saddle shape for conformingly fitting the intracavity bulge.

17. The probe of claim 16, further comprising a inflating device connected to the shaft member for inflating the inflatable balloon.

18. The probe of claim 17, further comprising a positioning scale printed on the outer surface thereof.

19. The probe of claim 18, further comprising anti-migration device for preventing unwanted movement of the probe relative to the cavity of the patient.

20. A system for planning and delivering radiation therapy treatment to an internal organ of a patient by a radiation emitting device, comprising:
a radiation emitting device; and
an insertable intracavity probe for immobilizing an internal organ within a cavity of a patient during delivery of a dose from the radiation emitting device, the probe comprising:
a shaft member having a distal tip; and
an interface device connected to the distal tip for conformingly fitting a bulge within the cavity corresponding to the internal organ to immobilize the internal organ during delivery of a dose from a radiation emitting device.

21. The system of claim 20, wherein the interface device comprises an inflatable balloon.

22. The system of claim 21, wherein the inflatable balloon comprises a surface having an anterior saddle shape for conformingly fitting the intracavity bulge.

23. The system of claim 22, further comprising a inflating device connected to the shaft member for inflating the inflatable balloon.

24. The system of claim 23, further comprising a positioning scale printed on the outer surface thereof.

25. The system of claim 24, further comprising anti-migration device for preventing unwanted movement of the probe relative to the cavity of the patient.

26. The method of claim 1, further comprising the step of using the radiation emitting device to provide a therapeutic dose of radiation.

27. The system of claim 23 wherein the inflating device comprises a syringe.

28. The system of claim 25 wherein the anti-migration device comprises a disk.

29. A method of localizing an internal organ of a patient comprising:
inserting a probe into a cavity of the patient;
acquiring an image of the patient in which the position of the probe is identifiable; and
localizing the internal organ based upon the position of the probe.

30. The method of claim 29, further comprising inflating an interface device of the probe into contact with the cavity after inserting the probe.

31. The method of claim 30, wherein the step of inserting the probe further comprises causing an anterior saddle shaped surface of the interface device to conformingly fit against a shaped portion of the cavity proximate the internal organ.

32. The method of claim 30, wherein the step of localizing the internal organ comprises identifying a volume of air defined by the inflatable interface device to approximate the location of the internal organ.

33. An insertable intracavity probe for localizing an internal organ within a cavity of a patient during, the probe comprising:
a shaft member having a distal tip;
an interface device connected to the distal tip for conformingly fitting a bulge within the cavity corresponding to the internal organ to localize the internal organ during delivery of an image dose from a radiation emitting device.

34. The probe of claim 33, wherein the interface device comprises an inflatable balloon.

35. The probe of claim 34, wherein the inflatable balloon defines a volume of air that is identifiable when subject to an image dose by the radiation emitting device to approximate the location of the internal organ.

36. The probe of claim 35, wherein the inflatable balloon comprises a surface having an anterior saddle shape for conformingly fitting the intracavity bulge.

37. The probe of claim 36, further comprising a inflating device connected to the shaft member for inflating the inflatable balloon.

38. The probe of claim 37, further comprising a positioning scale printed on the outer surface thereof.

39. The probe of claim 38, further comprising anti-migration device for preventing unwanted movement of the probe relative to the cavity of the patient.

40. A method of localizing an internal organ of a patient, comprising:
inserting a probe into the patient, the probe comprising a shaft having a distal tip and an interface device operably associated with the distal tip for conformingly fitting at least a portion of the internal organ; and
localizing the internal organ for delivery of a dose from a radiation emitting device.
